(19) United States
(12) Patent Application Publication
(10) Pub. No.: US 2021/0385935 A1
(43) Pub. Date: Dec. 9, 2021

(54) PLASMA TREATMENT DEVICE

(71) Applicant: CINOGY GMBH, Duderstadt (DE)

(72) Inventors: Dirk WANDKE, Heiligenstadt (DE); Mirko HAHNL, Berlingerode (DE); Karl-Otto STORCK, Duderstadt (DE); Leonhard TRUTWIG, Duderstadt/Gerblingerode (DE); Melanie RICKE, Katlenburg-Lindau (DE)

(21) Appl. No.: 17/287,708

(22) PCT Filed: Oct. 23, 2019
(86) PCT No.: PCT/EP2019/078894

§ 371 Date: Apr. 22, 2021

(30) Foreign Application Priority Data
Oct. 24, 2018 (DE) 10 2018 126 492.5

(51) Int. Cl. H05H 1/24; A61N 1/32; A61N 1/04; A61L 2/14; A61L 2/00

(52) U.S. Cl. CPC H05H 1/2418; A61N 1/322; A61N 1/0408; H05H 2245/60; A61L 2/14; A61L 2/0011; H05H 2245/34; A61N 1/0472

(57) ABSTRACT

A plasma treatment device (1) designed to treat a surface with a dielectrically impeded plasma, having a base body (3) that has at least one flat treatment side (5) facing the surface to be treated, and having an electrode arrangement (9) that has at least one electrode (7) and having a dielectric that completely covers the at least one electrode (7) in the direction of the surface to be treated, and having a line arrangement comprising at least one high-voltage supply line (13, 13a, 13b), wherein the electrode (7) is connected to the line arrangement and is able to be supplied, via the high-voltage supply line (13, 13a, 13b), with a high-voltage signal able to be applied to the high-voltage supply line (13, 13a, 13b), and having a nub arrangement (15) arranged on the treatment side (5) of the base body (3) and that has a multiplicity of nubs (17), makes it possible to easily combine an effective plasma treatment with an effective mechanical treatment of the surface to be treated in that the at least one electrode (7) of the electrode arrangement (9) extends into at least one nub (17) of the nub arrangement (15).

The invention relates to a plasma treatment device designed to treat a surface with a dielectrically impeded plasma, having a base body that has at least one flat treatment side facing the surface to be treated, and having an electrode arrangement that has at least one electrode and having a dielectric that completely covers the at least one electrode in the direction of the surface to be treated, and having a line arrangement comprising at least one high-voltage supply line, wherein the electrode is connected to the line arrangement and is able to be supplied, via the high-voltage supply line, with a high-voltage signal able to be applied to the high-voltage supply line, and having a nub arrangement arranged on the treatment side of the base body and that has a multiplicity of nubs.

A treatment side in the context of the present application is understood to mean a side that faces the surface to be treated when the plasma treatment device is used as intended. In contrast thereto, a side facing away from the surface to be treated in the context of the present application is understood to mean a side that faces away from the surface to be treated, that is to say does not face the surface to be treated, when the plasma treatment device is used as intended.

The electrode arrangement of the plasma treatment device may advantageously in particular be configured such that the surface to be treated is used as counter-electrode. To this end, the surface to be treated has to be the surface of an electrically conductive body. Such an electrically conductive body may be for example a human or animal body or another electrically conductive body.

The surface to be treated or the associated body may in this case function as what is known as a floating counter-electrode. Such a floating counter-electrode follows the change in potential of the high-voltage signal applied to the electrode only very slowly. If an AC high-voltage signal is used as high-voltage signal, then the potential of the floating counter-electrode remains substantially at an average potential, which will generally be the reference potential. The surface to be treated or the associated body may therefore function as ground.

By way of example, it is possible to use only a single electrode to generate the plasma and to use the surface or the associated body as counter-electrode (ground). This advantageously achieves a large treatment depth within the body.

It is also possible for example to provide a plurality of electrodes, that is to say at least two electrodes, in the electrode arrangement, which are supplied with the same high-voltage signal. The surface to be treated in this case functions as counter-electrode for the plasma formation. As an alternative, it is possible for example to supply the at least two electrodes of the electrode arrangement with different AC high-voltage signals, in particular with AC high-voltage signals in phase opposition to one another. In this case too, the surface to be treated may again function as counter-electrode.

However, it is also possible for example to use the at least two electrodes of the plurality of electrodes as electrode and counter-electrode, such that the plasma arises between the electrodes and is able to act as surface plasma. Only relatively small treatment depths are thereby possible with a normal energy input, however.

For a plasma treatment device of the type mentioned at the outset, it is essential for the dielectric to completely cover the at least one electrode in the direction of the surface to be treated. The at least one electrode is thereby shielded from the surface to be treated. Due to the fact that the dielectric completely covers the at least one electrode in the direction of the surface to be treated, a direct or galvanic current flow between the at least one electrode and the surface to be treated is prevented.

The dielectric or parts of the dielectric may in particular be designed to be flexible. Suitable materials for producing the flexible dielectric are for example flexible silicones, in particular silicone rubbers. However, it is also conceivable for example to use other electrically non-conductive plastics, for example to use thermoplastic elastomers (for example thermoplastic polyethylene).

The base body and/or the nub arrangement, in particular the nubs of the nub arrangement, may advantageously likewise be designed to be completely or partially flexible. Suitable materials for producing the flexible nub arrangement are likewise for example flexible silicones, in particular silicone rubbers. However, it is also conceivable here to use other plastics, for example to use thermoplastic elastomers (for example thermoplastic polyethylene).

DE 10 2015 111 401 B3 discloses a treatment device for treating a surface with a dielectrically impeded plasma. The treatment device has a housing having an end wall and an electrode. The electrode is able to be connected to a high-voltage generator and is shielded in the direction of the surface to be treated by a dielectric forming at least part of the end wall. The end wall has at least one spacer that forms at least one gas compartment when the spacer bears on the surface to be treated, in which gas compartment the dielectrically impeded plasma for the treatment is formed.

DE 10 2012 015 482 A1 furthermore discloses an electrode arrangement for forming a dielectrically impeded plasma between an active surface of the electrode arrangement and a surface functioning as counter-electrode. This electrode arrangement has a flexible, flat electrode that is able to be connected to a high-voltage source, and a flat, flexible dielectric that forms the active surface and that is connected to the flat electrode so as to form an electrode element and completely covers the electrode in the direction of the surface to be treated. In order to improve adaptability to uneven surfaces, this electrode arrangement furthermore has a surface-elastic pressure means on the rear side of the electrode element that faces away from the surface, by way of which the flat electrode element is pressed evenly in the direction of the surface to be treated.

The possible uses of such devices for treating surfaces with a dielectrically impeded plasma are extremely varied. They lie in particular in the therapeutic and cosmetic sector, but are by no means restricted thereto. Known devices for treating surfaces with a dielectrically impeded plasma have proven themselves and are suitable in particular for treating the skin surface of a human or animal body. The plasma treatment may for example improve the take-up of therapeutic or cosmetic active ingredients, such that the plasma treatment enhances the desired therapeutic or cosmetic effect. The plasma treatment furthermore ensures effective germ reduction, since it destroys microorganisms and in particular exerts a bactericidal and fungicidal effect on the skin. The plasma treatment also leads to an increase in microcirculation in tissue.

In many of the diverse applications of such plasma treatments, it is desirable to be able to combine the plasma treatment with a mechanical treatment of the surface, for example in order to achieve a massage and/or cleaning effect. This is of particular interest in the plasma treatment of a human or animal body, for example in the plasma treatment of the skin of a human or the skin of an animal or the hide of an animal. The abovementioned previously known plasma treatment devices however do not allow such a mechanical treatment of a surface in order to achieve a massage and/or cleaning effect.

Taking this as a starting point, the present invention is based on the object of improving the proven, previously known plasma treatment devices such that they allow both an effective plasma treatment and an effective mechanical treatment of the surface to be treated, in particular a surface of a human or animal body to be treated, in order to achieve a massage and/or cleaning effect.

In order to achieve this object, a plasma treatment device of the type mentioned at the outset is characterized, according to the invention, in that the at least one electrode of the electrode arrangement extends into at least one nub of the nub arrangement.

In this case, the at least one electrode of the electrode arrangement may advantageously in particular extend into a plurality or a multiplicity of nubs of the nub arrangement and/or into all of the nubs of the nub arrangement. In one embodiment of the invention, all of the nubs of the nub arrangement may accordingly be nubs into which an electrode of the electrode arrangement extends. In other embodiments of the invention, the nub arrangement may both have nubs into which an electrode of the electrode arrangement extends and have nubs into which no electrode extends. The plasma treatment device according to the invention, by virtue of its nub structure into the nubs of which the electrode extends, advantageously allows a plasma treatment to be combined with a mechanical treatment of the surface to be treated. The plasma treatment may thereby be integrated into an operation for example involving cleaning and/or massaging of the surface to be treated.

The plasma treatment and the mechanical treatment of the surface to be treated with the plasma treatment device according to the invention may in this case be performed simultaneously. The plasma treatment and the mechanical treatment may however in principle also take place in succession in any chronological order. The plasma treatment device may accordingly be designed to perform a plasma treatment and a mechanical treatment of the surface to be treated simultaneously and/or in succession.

One exemplary field of application of the plasma treatment device according to the invention is the therapeutic and/or cosmetic treatment of a human or animal body. By way of example, it is advantageously possible to clean and/or to massage the body surface in connection with a plasma treatment of a body surface of a human or animal body. By massaging the body surface, it is possible in the process to act therapeutically on the muscles and/or the connective tissue and/or the skin of the human or animal body. The mechanical treatment of a skin surface of a human or animal body with the plasma treatment device according to the invention may in particular for example be used to stimulate blood flow. The plasma treatment device according to the invention may advantageously also be used for example to combine a plasma treatment with peeling of the skin.

The mechanical treatment of the surface may however also be used to clean the surface, for example to clean a human or animal skin or an animal hide.

The mechanical treatment of the surface to be treated may also be used to improve the effectiveness of the plasma treatment, for example by virtue of opening the pores of the surface to be treated through the mechanical action. When treating a skin surface, stimulating blood flow may contribute to improving the effectiveness of the plasma treatment.

The treatment of the surface to be treated with the plasma treatment device according to the invention may advantageously be performed using therapeutic and/or cosmetic active ingredients. The treatment of the surface to be treated with the plasma treatment device according to the invention may advantageously be performed for example using a cleansing cream and/or a care cream. The treatment of the surface to be treated may however advantageously also be performed dry and/or without using therapeutic or cosmetic active ingredients, cleansing creams or care creams.

In addition to effective mechanical treatment, the plasma treatment device according to the invention advantageously achieves an effective plasma treatment of the surface to be treated due to the fact that the at least one electrode of the electrode arrangement extends into one or more nubs of the nub arrangement. During intended use of the plasma treatment device, that is to say during the treatment of the surface, it is thereby possible to guarantee a small distance between the electrode extending into one or more nubs and the surface to be treated, regardless of the length of the nubs. By virtue of the small distance between the at least one electrode and the surface to be treated, effective formation of the plasma in the area of the surface to be treated and a large treatment depth are advantageously achieved.

The nub arrangement of the plasma treatment device according to the invention may furthermore advantageously be used to ensure that air spaces in which the plasma is able to form are maintained in the intermediate spaces between the nubs of the nub arrangement during the treatment. Such air spaces are essential for the desired effective formation of the plasma, and thus for an effective plasma treatment.

In one advantageous embodiment, the electrode arrangement of the plasma treatment device according to the invention may in particular have a plurality of electrodes that each extend in the described manner into at least one nub of the nub arrangement. The different electrodes of the plurality of electrodes may in this case be electrically conductively connected to one another and/or connected to the same electrical potential. The different electrodes of the plurality of electrodes may however also be galvanically isolated from one another and/or connected to different electrical potentials.

The nubs of the nub arrangement of the plasma treatment device according to the invention may advantageously be designed to be substantially circular-cylindrical, substantially conical and/or substantially frustoconical. The nubs may in particular be designed to be substantially circular-cylindrical with a rounded tip, in particular with a substantially hemispherical tip. The nubs may however also be designed to be substantially cuboidal, substantially pyramidal and/or substantially frusto-pyramidal. The nubs may however in principle also have the shape of another geometric body.

The cross section of the individual nubs may in particular be substantially circular and/or elliptical and/or substantially square. The cross section of the individual nubs may advantageously have a longest and a shortest side, wherein the longest side has a length that is no more than two and a half times, in particular no more than twice, in particular no more than one and a half times the length of the shortest side.

The nubs of the nub arrangement may in particular be of elongate design. The ratio of the respective length of the nubs to the longest dimension of their respective cross section may advantageously be greater than 1, in particular greater than 1.5, in particular greater than 2, in particular greater than 3, in particular greater than 4, in particular greater than 5, in particular greater than 6, in particular greater than 8 and in particular greater than 10.

The geometric shapes and in particular the lengths of the individual nubs of the nub arrangement may be designed to be identical to or different from one another.

In one advantageous development of the invention, the electrode arrangement may in particular have at least two electrodes or at least three electrodes or at least four electrodes or at least five electrodes or at least six electrodes or at least eight electrodes or at least ten electrodes or at least 15 electrodes or at least 20 electrodes or at least 25 electrodes or at least 30 electrodes or at least 40 electrodes or at least 50 electrodes, each of which extends into at least one nub of the nub arrangement.

In a further advantageous development of the invention, provision is made for the nub arrangement to be formed partially or completely from the dielectric. The nubs of the nub arrangement may in this case in particular be formed partially or completely from the dielectric. As an alternative or in addition thereto, the base body may advantageously be formed partially or completely from the dielectric.

Such developments of the invention, in which the nub arrangement and/or the base body are formed partially or completely from the dielectric, offer the advantages of a particularly simple structure and thus particularly simple production of the plasma treatment device according to the invention.

In a further advantageous development of the invention, provision is made for the nub arrangement to be designed in one piece, that is to say produced in one piece. This offers the advantage that the nub arrangement is able to be produced in a particularly simple and inexpensive manner, for example in a casting process or in a 3D printing process.

In a further advantageous development of the invention, provision is made for the nub arrangement to be designed in one piece with the base body. Such an embodiment of the plasma treatment device according to the invention is particularly advantageous if the nub arrangement and the base body are formed from the dielectric.

Such developments of the plasma treatment device according to the invention, in which the nub arrangement is formed in one piece with the base body, offer the advantage that the one-piece arrangement of the base body and the nub arrangement is able to be produced in a particularly simple and inexpensive manner in a casting process. In this case, the at least one electrode of the electrode arrangement, which extends into at least one nub of the nub arrangement, is able to be easily cast. It is however likewise also advantageously possible to quickly build a prototype in a 3D printing process.

As an alternative, it is however also possible for the nub arrangement to be produced as a separate part in order then to be affixed to the base body. A fixed connection may in this case be created in the usual manner, that is to say positively and/or substance-to-substance, in particular through adhesive bonding and/or welding. It is possible in this case for the at least one electrode of the electrode arrangement to be cast into the nub arrangement and/or the base body.

In a further advantageous development of the invention, provision is made for the at least one electrode of the electrode arrangement to consist of a castable plastic provided with conductive additives. The at least one electrode of the electrode arrangement that extends into at least one nub of the nub arrangement may advantageously in this case in particular consist of a castable plastic provided with conductive additives.

The conductive additives create electrical conductivity in the plastic, such that it is able to be used to produce the electrode. The castable plastic may in this case in particular be a silicone. Suitable conductive additives are for example metal particles, carbon particles or the like.

Such a development of the plasma treatment device according to the invention having an electrode made of a castable plastic provided with conductive additives offers the advantage of allowing a substance-to-substance (that is to say material) bond between the electrode and a dielectric that is likewise made of plastic and covers the electrode. The substance-to-substance bond in this case results from the plastics themselves, and does not require any additional adhesive layer at the boundary layer between the electrode and the dielectric. The electrode and the dielectric covering the electrode may thus be formed as a single material, as it were, and thus form a particularly durable connection. This is particularly important because the electrode extending into a nub may be subjected to severe bending during the treatment.

This is particularly advantageous when the electrode of the electrode arrangement, consisting of a castable plastic provided with conductive additives, extends into at least one nub that is formed partially or completely from the dielectric. This results specifically in a particularly simple and therefore inexpensive-to-produce embodiment of the plasma treatment device according to the invention.

In a further advantageous development of the invention, provision is made for the at least one electrode of the electrode arrangement to have a flexible design. For this purpose, the at least one electrode of the electrode arrangement may consist of a flexible and electrically conductive material. The flexibility may in this case result from an elastic deformability, but also from a plastic deformability of the material of the electrode. For this purpose, the at least one flexible electrode of the electrode arrangement may be made for example of a conductive and flexible plastic, a conductive plastic powder, conductive plastic granules, a metal powder and/or metal granules. Electrically conductive silicone may in particular serve as electrically conductive plastic. The electrical conductivity of the plastic may in this case be generated in the manner described above by virtue of providing the plastic with conductive additives.

Such an embodiment of the plasma treatment device according to the invention, in which the at least one electrode has a flexible design, offers the advantage that the electrode is able to adapt its shape to the surface to be treated during the treatment. This achieves a particularly high level of effectiveness both for the plasma treatment and for the mechanical treatment. This is particularly advantageous if the nub arrangement or at least the nubs of the nub arrangement into which the at least one electrode extends are also designed to be flexible.

In a further advantageous embodiment of the invention, the at least one electrode of the electrode arrangement may however also have a rigid design.

It is of course also possible for the electrode arrangement to have a plurality of electrodes and to comprise both flexible and rigid electrodes. The electrode arrangement may accordingly have at least one flexible electrode and/or at least one rigid electrode.

In a further advantageous development of the invention, provision is made for the electrode arrangement to be at least partially embedded in the base body. The at least one electrode of the electrode arrangement, which extends into at least one nub of the nub arrangement, may in this case protrude from the base body on the treatment side. The at least one electrode protruding from the base body may in this case protrude into at least one nub of the nub arrangement and thereby extend into the nub.

Such an embodiment of the invention, in which the electrode arrangement is at least partially embedded in the base body, offers the advantage that it is thereby easily possible to ensure that the at least one electrode is shielded from the surface to be treated. A particularly simple structure of the plasma treatment device results in this case when the base body is formed at least partially from the dielectric.

In a further advantageous development of the invention, provision is made for the electrode arrangement to have a plurality of finger-shaped electrodes that are surrounded by a dielectric layer and each extend into a nub. The finger-shaped electrodes and the dielectric layer surrounding them may in particular in this case be designed to be flexible. The nubs into which the finger-shaped electrodes extend may advantageously in this case in particular also be formed from the dielectric, in particular from a dielectric with a flexible design.

The cross section of the individual finger-shaped electrodes may in this case in particular be substantially circular and/or elliptical and/or substantially square. The cross section of the individual finger-shaped electrodes may advantageously have a longest and a shortest side, wherein the longest side has a length that is no more than two and a half times, in particular no more than twice, in particular no more than one and a half times the length of the shortest side.

Such embodiments of the plasma treatment device according to the invention, which have a plurality of finger-shaped electrodes, offer the advantage that it is possible to implement an electrode arrangement having a multiplicity of electrodes that each extend into a nub of the nub arrangement. The electrodes that each extend into a nub may thereby be distributed over the entire surface of the nub arrangement. This offers the advantage of a plasma treatment that is distributed particularly evenly over the surface and is therefore particularly effective.

In a further advantageous development of the invention, provision is made for the plasma treatment device to have at least one handle for holding the plasma treatment device. As an alternative or in addition thereto, the plasma treatment device may have at least one strap for holding the plasma treatment device. The plasma treatment device may advantageously have at least one handle and/or at least one strap for holding the plasma treatment device, in particular on a side facing away from the surface to be treated. The side facing away from the surface to be treated may in this case advantageously in particular be a side opposite the treatment side, that is to say the at least one handle and/or the at least one strap may advantageously be arranged in particular on a side opposite the treatment side.

The handle and/or the strap may in this case in principle have any shape that is suitable for holding the plasma treatment device. The handle may advantageously in particular be designed as a hand-operated handle, that is to say as a handle able to be grasped by a human hand. The strap may in this case advantageously be designed in particular as a hand strap, that is to say as a strap able to be grasped by a human hand and/or into which a human hand is able to be inserted so as to hold the plasma treatment device. Other types of handle and/or strap that are suitable for holding the plasma treatment device are however also conceivable.

Such a development of the invention offers the advantage that the plasma treatment device is thereby particularly well-suited to manual treatment of a surface, for example to manual cleaning or massage of a human or animal body. For this purpose, the plasma treatment device may for example be gripped with one or two hands on the at least one handle and/or the at least one strap and guided over the surface to be treated while exerting contact pressure.

In a further advantageous development, provision is made for the plasma treatment device to be in the form of a curry comb.

It is therefore proposed for the plasma treatment device according to the invention to be designed to treat the hide of an animal. Such a development of the invention offers the advantage of being able to combine the care and/or cleaning of the animal hide with a plasma treatment and the positive effects associated therewith (see above).

In a further advantageous development of the invention, provision is made for the plasma treatment device to be designed as a floor mat on which at least one foot is placed. As an alternative or in addition thereto, the plasma treatment device may be designed as a body part support on which at least one body part is placed. The body part support mat on which at least one body part is placed may in particular be a hand support mat on which at least one hand is placed.

For this purpose, the plasma treatment device may be designed to be placed on a surface with a bottom side. The bottom side of the plasma treatment device is in this case a side facing away from the surface to be treated, and may in particular be a side opposite the treatment side. The plasma treatment device may in this case advantageously be designed in particular to be placed in a non-slip manner on a surface with the bottom side and, for this purpose, to have for example a non-slip coating and/or a non-slip structure on the bottom side.

Such a development of the invention offers the advantage that the plasma treatment device according to the invention is able to be used in the manner of a body massage mat that is known per se, for example in the manner of a foot massage mat that is known per se. The plasma treatment may thereby advantageously be combined with the massage effect able to be achieved by the nubs of the nub arrangement.

In a further advantageous development of the invention, provision is made for the base body to be designed in the form of a glove and to have an insertion opening through which a human hand is able to be inserted into the glove. The glove may in this case in particular be a glove with fingers.

It is thereby possible, to perform the treatment, for a hand to be inserted into the base body, which is designed as a glove, and for the plasma treatment device to be guided over the surface to be treated by way of the hand inserted into the base body. Such a development of the invention offers the advantage that the plasma treatment device is suitable for a particularly effective manual treatment of a surface, for example for cleaning and/or massaging a human or animal body. The design of the base body in the form of a glove in this case advantageously allows the movement of the plasma treatment device during the treatment, and in particular the contact pressure exerted in the process, to be able to be controlled particularly well. This embodiment of the invention is particularly advantageous if the base body, which is designed as a glove, and/or the nub arrangement are designed to be flexible in the manner explained above. In this case, a particularly simple and effective treatment of uneven and/or irregularly shaped surfaces to be treated is made possible.

In such an embodiment of the invention, in which the base body is designed in the form of a glove, the plasma treatment device may advantageously be designed in particular to treat a skin surface of a human and/or animal body and/or to treat the hide of an animal.

In a further advantageous development of the invention, provision is made for the plasma treatment device to have an electrically insulating housing in which at least part of the at least one high-voltage supply line of the line arrangement is arranged.

In this case, the housing may advantageously be arranged in particular next to the nub arrangement on the treatment side of the base body. This offers the advantage of providing a simple structural solution for embodiments of the plasma treatment device in which the plasma treatment device is intended to be placed on a surface with a bottom side facing away from the surface to be treated. By way of example, such a development of the invention is thus particularly suitable for embodiments in which the plasma treatment device is designed, in the manner described above, as a floor mat and/or as a body part support mat, in particular as a hand support mat.

In a further advantageous development of the invention, provision is made for the housing to be designed in at least two parts and to have a lower housing part and an upper housing part designed in the form of a cover and that is able to be placed on the lower housing part. This offers the advantage that it is easily possible to access the components of the plasma treatment device that are arranged in the housing, for example for maintenance or repair purposes, for connecting a connection cable for a voltage supply or for inserting or changing a battery.

It is also conceivable for the housing to be arranged in the base body or be formed in the base body. The housing may also be formed entirely or partially from the base body.

In a further advantageous development of the invention, provision is made for at least one part of the housing, which may in particular be a lower housing part of an at least two-part housing of the type described above, to be formed by the base body of the plasma treatment device. This offers the advantage of a particularly simple structural implementation of a plasma treatment device according to the invention with a housing of the type described above. For this purpose, a lower housing part may for example simply be formed in the base body, onto which an upper housing part designed in the form of a cover is able to be placed.

The plasma treatment device according to the invention may be designed to be connected to an external high-voltage source. However, it is preferable for the plasma treatment device to be designed to generate the high voltage in the plasma treatment device itself. A normal supply voltage (for example 230 V or 110 V mains voltage) may in this case be supplied to the plasma treatment device.

In a further advantageous development of the plasma treatment device according to the invention, there is therefore provision for the plasma treatment device to have a high-voltage stage for generating high-voltage signals suitable for generating the plasma and that is connected at output to the at least one high-voltage supply line of the line arrangement. The high-voltage stage for generating the high-voltage signals may in this case in particular be arranged in the housing of the type described above. The high-voltage stage may for example be a high-voltage generator.

Such a development of the plasma treatment device according to the invention offers the advantage that no high voltage has to be carried outside the device, such that it is significantly easier to guarantee the required safety of the device.

The housing and/or the base body of the plasma treatment device according to the invention may have a through-opening for the passage of a connection cable. The plasma treatment device may be supplied with a high voltage or, preferably, with a normal supply voltage of the abovementioned type via this connection cable.

In a further advantageous development of the invention, provision is made for the plasma treatment device to have at least one battery for providing a supply voltage required for the operation of the plasma treatment device. The at least one battery may in this case in particular be arranged in a housing of the type described above. The battery may be a rechargeable battery (accumulator) or a non-rechargeable battery. The high-voltage stage is able to generate the required high voltage from the supply voltage provided by the battery.

Such a development of the invention offers the advantage that the plasma treatment device is able to be designed without wires, and is thereby able to be used in a particularly flexible and mobile manner.

In a further advantageous development of the invention, provision is made for the electrode arrangement to comprise a plurality of electrodes and to have at least one electrically conductive distributor plate. The distributor plate is in this case connected to the high-voltage supply line and a plurality of electrodes and is configured to distribute the high-voltage signal to the electrodes connected to the distributor plate.

Such an embodiment of the plasma treatment device according to the invention having a distributor plate offers the advantage of a low-loss and at the same time structurally easy-to-implement distribution of the high-voltage signal to the plurality of electrodes of the electrode arrangement.

In a further advantageous development of the invention, provision is made for the line arrangement to comprise a plurality of high-voltage supply lines to which different high-voltage signals are able to be applied, and for the electrode arrangement to comprise a plurality of electrodes that form a plurality of electrode groups that are electrically insulated from one another. The different electrode groups are in this case connected to different high-voltage supply lines and are able to be supplied with different high-voltage signals.

It is therefore proposed for the electrode arrangement to have a plurality of electrode groups that are able to be supplied with different high-voltage signals. In the simplest case, the electrode arrangement may in this case have two electrodes that are electrically insulated from one another and each form an electrode group consisting of just one electrode.

The different electrode groups may advantageously in this case be supplied with different high-voltage signals that are of opposing polarity to one another. The different high-voltage signals may in this case in particular be of opposing polarity and the same absolute value. The high-voltage signals may advantageously in particular be in the form of AC high-voltage signals, and the different electrode groups may be supplied with AC high-voltage signals in phase opposition. The AC high-voltage signals in phase opposition may in this case in particular have a substantially identical amplitude.

Such an embodiment of the plasma treatment device according to the invention offers the advantage that the resulting electric fields are destructively superimposed in their area of overlap and cancel one another out at some distance from the electrodes, such that the electric field required for plasma formation remains limited to the close area relevant to the plasma treatment. This furthermore offers the advantage of being able to avoid undesired field peaks, which could result from constructive superimposition of the electric fields generated by the different electrode groups in their area of overlap.

In a further advantageous development of the invention, provision is made for the electrode arrangement to have a plurality of distributor plates that are electrically insulated from one another, wherein the electrodes of the different electrode groups are connected to different distributor plates and the different distributor plates are connected to different high-voltage supply lines.

It is thus proposed for the electrode arrangement to have a plurality of electrically conductive distributor plates that are electrically insulated from one another, wherein each distributor plate is connected to a plurality of electrodes and to one of the high-voltage supply lines and is configured to distribute the high-voltage signal to the electrodes connected to the distributor plate, and wherein the electrodes of the different electrode groups are connected to different distributor plates and the different distributor plates are connected to different high-voltage supply lines.

Such a development of the plasma treatment device according to the invention having a plurality of distributor plates electrically insulated from one another offers the advantage that it enables low-loss and at the same time easy-to-implement distribution of different high-voltage signals to different electrode groups.

The invention is intended to be explained in more detail below with reference to the exemplary embodiments illustrated schematically in the accompanying drawings. In the figures:

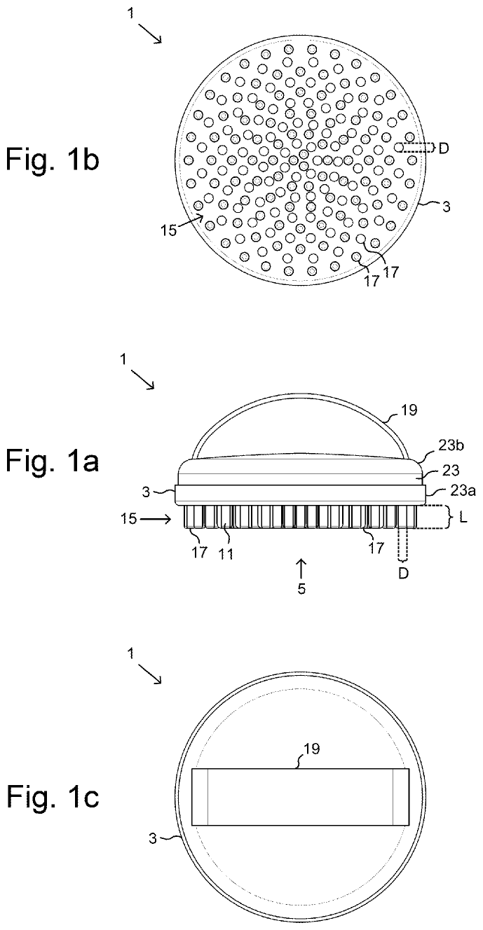

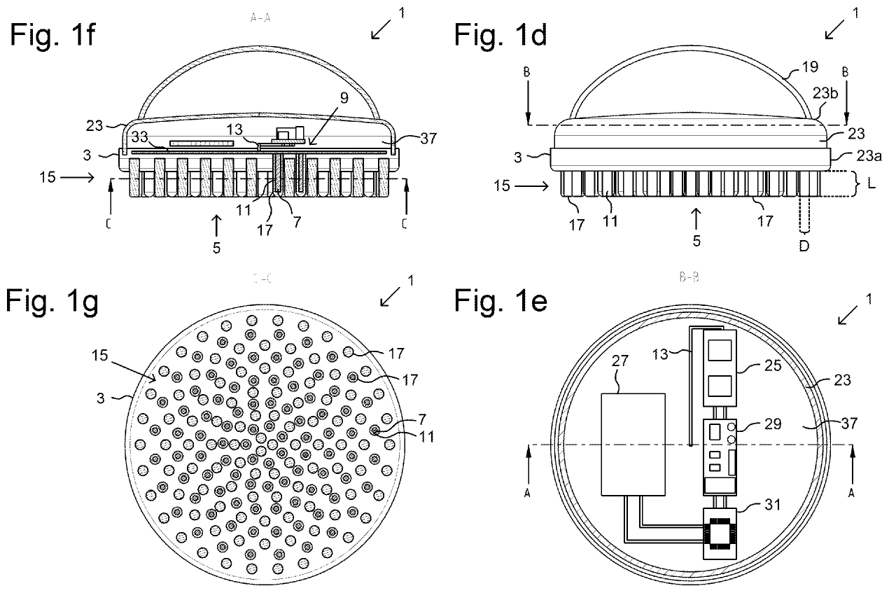

FIG. 1a shows a side view of a first embodiment of the plasma treatment device according to the invention, in which the plasma treatment device is in the form of a curry comb;

FIG. 1b shows a view of a treatment side of the first embodiment of the plasma treatment device;

FIG. 1c shows a view of an upper side of the first embodiment of the plasma treatment device;

FIG. 1d shows a further side view of the first embodiment of the plasma treatment device;

FIG. 1e shows a horizontal section along the line B-B in FIG. 1d;

FIG. 1f shows a vertical section along the line A-A in FIG. 1e;

FIG. 1g shows a horizontal section along the line C-C in FIG. 1f;

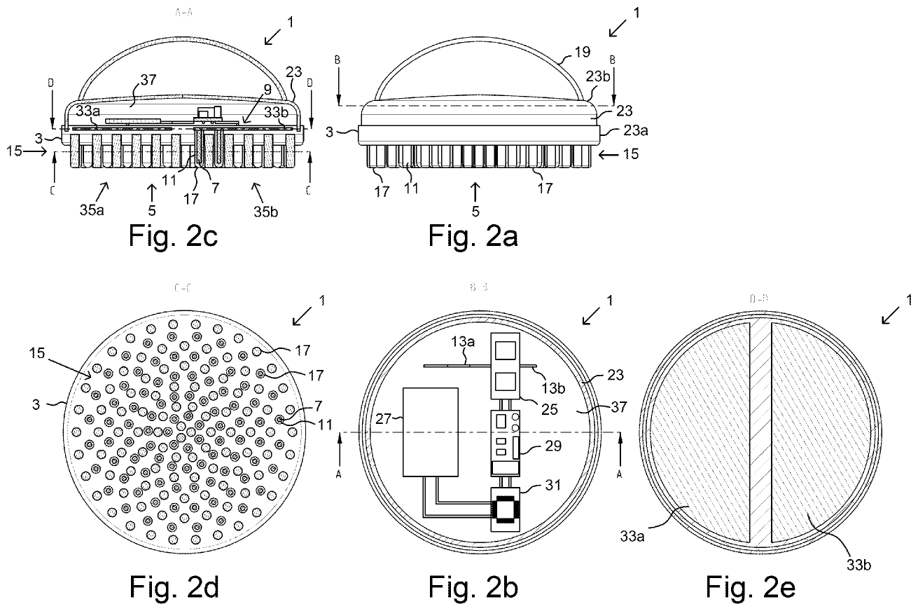

FIG. 2a shows a side view of a second embodiment of the plasma treatment device according to the invention, in which the plasma treatment device is in the form of a curry comb;

FIG. 2b shows a horizontal section along the line B-B in FIG. 2a;

FIG. 2c shows a vertical section along the line A-A in FIG. 2b;

FIG. 2d shows a horizontal section along the line C-C in FIG. 2c;

FIG. 2e shows a horizontal section along the line D-D in FIG. 2c;

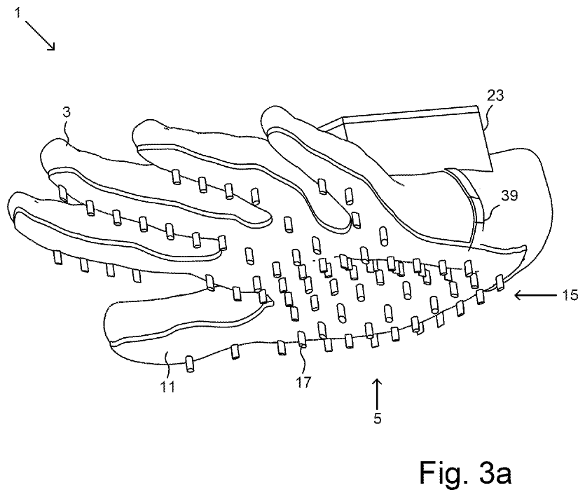

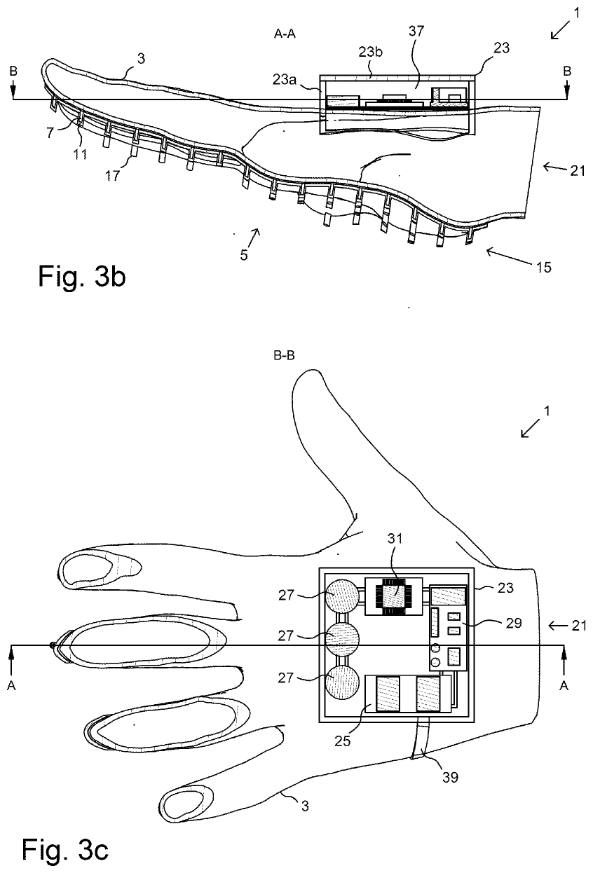

FIG. 3a shows a perspective view of a third embodiment of the plasma treatment device according to the invention, having a base body designed in the form of a glove;

FIG. 3b shows a sectional illustration of the third embodiment in the form of a vertical section along the line A-A in FIG. 3c;

FIG. 3c shows a sectional illustration of the third embodiment in the form of a horizontal section along the line B-B in FIG. 3b;

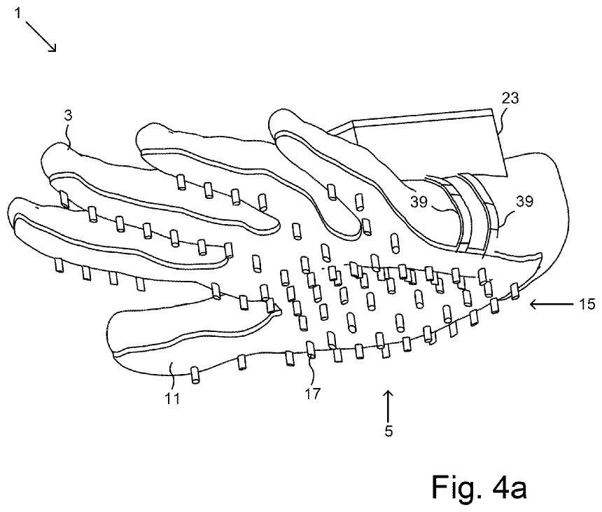

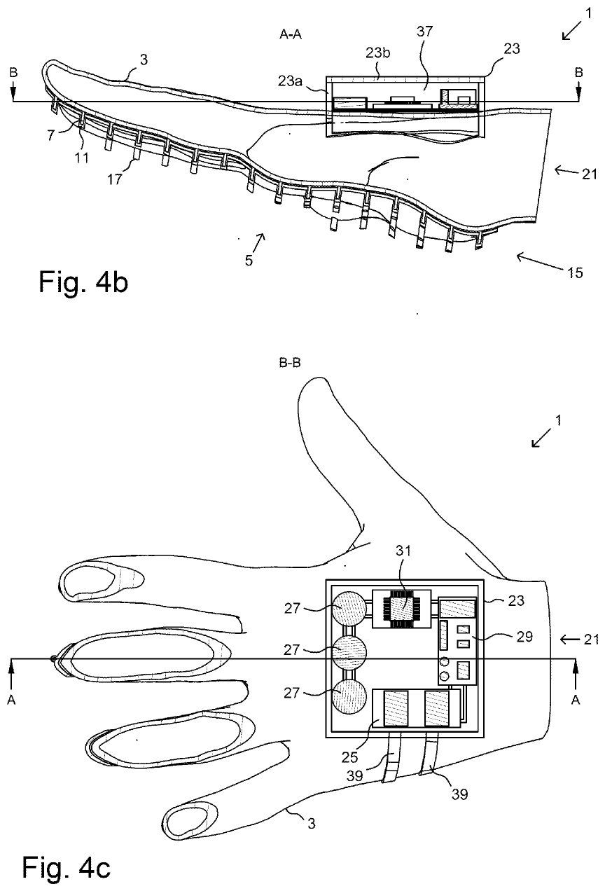

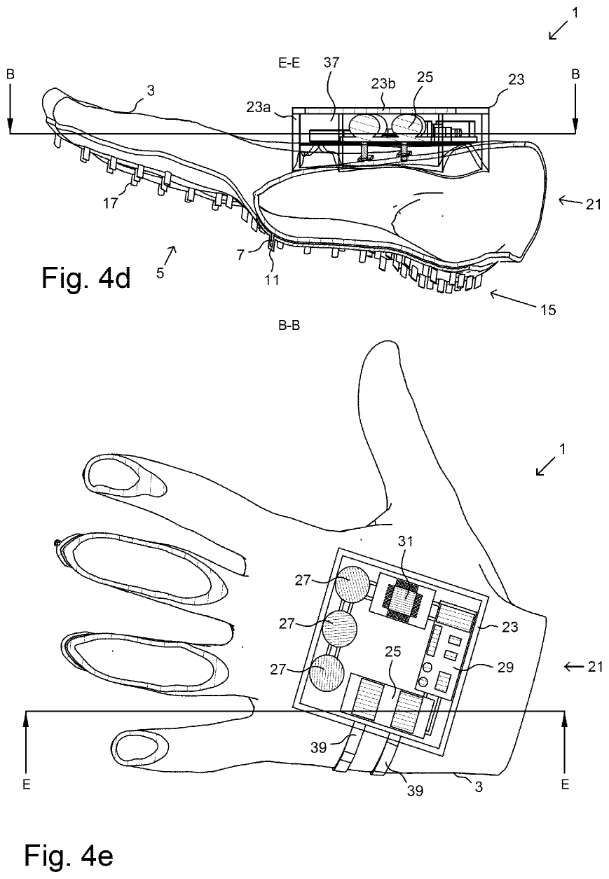

FIG. 4a shows a perspective view of a fourth embodiment of the plasma treatment device according to the invention, having a base body designed in the form of a glove;

FIG. 4b shows a sectional illustration of the fourth embodiment in the form of a vertical section along the line A-A in FIG. 4c;

FIG. 4c shows a sectional illustration of the fourth embodiment in the form of a horizontal section along the line B-B in FIG. 4b;

FIG. 4d shows a sectional illustration of the fourth embodiment in the form of a vertical section along the line E-E in FIG. 4e;

FIG. 4e shows a sectional illustration of the fourth embodiment in the form of a horizontal section along the line B-B in FIG. 4d;

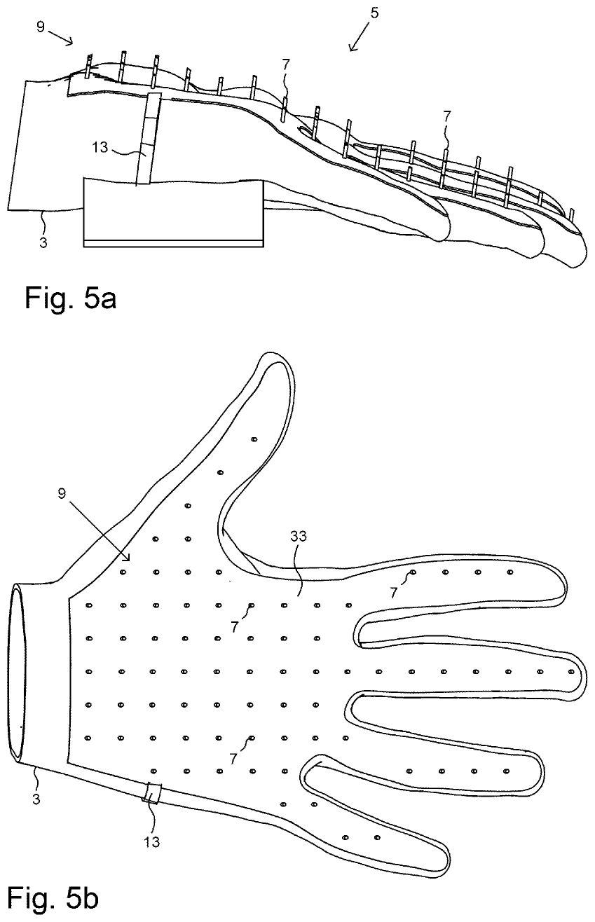

FIG. 5a shows a side view of components of the third embodiment of the plasma treatment device according to the invention;

FIG. 5b shows a view of a treatment side of the components of the third embodiment of the plasma treatment device according to the invention;

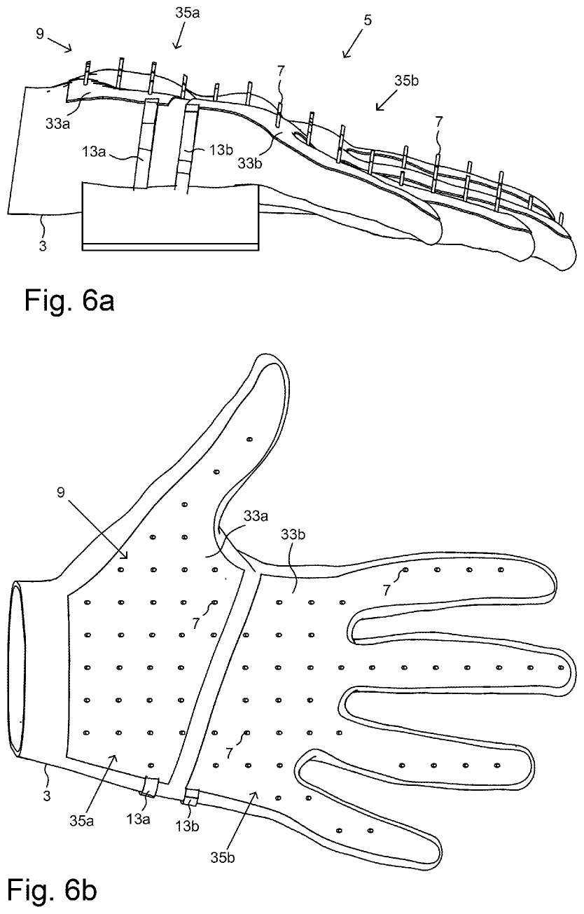

FIG. 6a shows a side view of components of the fourth embodiment of the plasma treatment device according to the invention;

FIG. 6b shows a view of a treatment side of the components of the fourth embodiment of the plasma treatment device according to the invention;

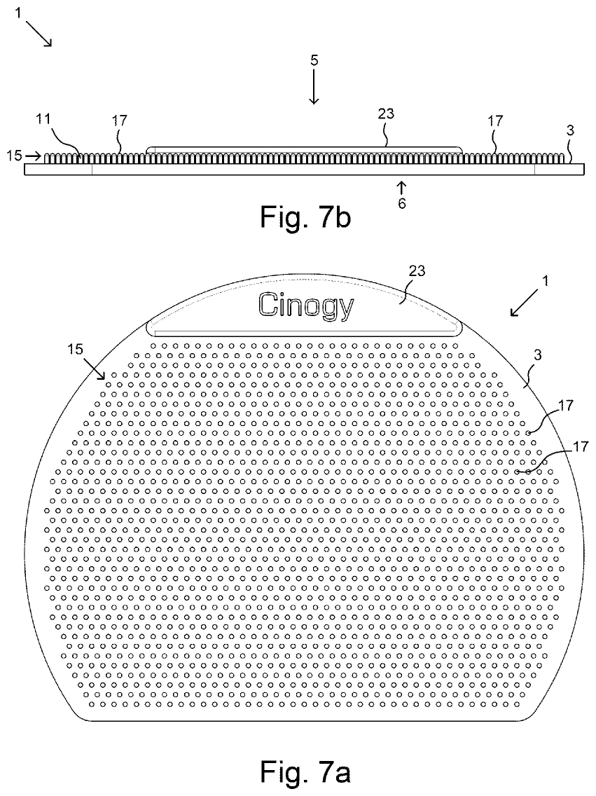

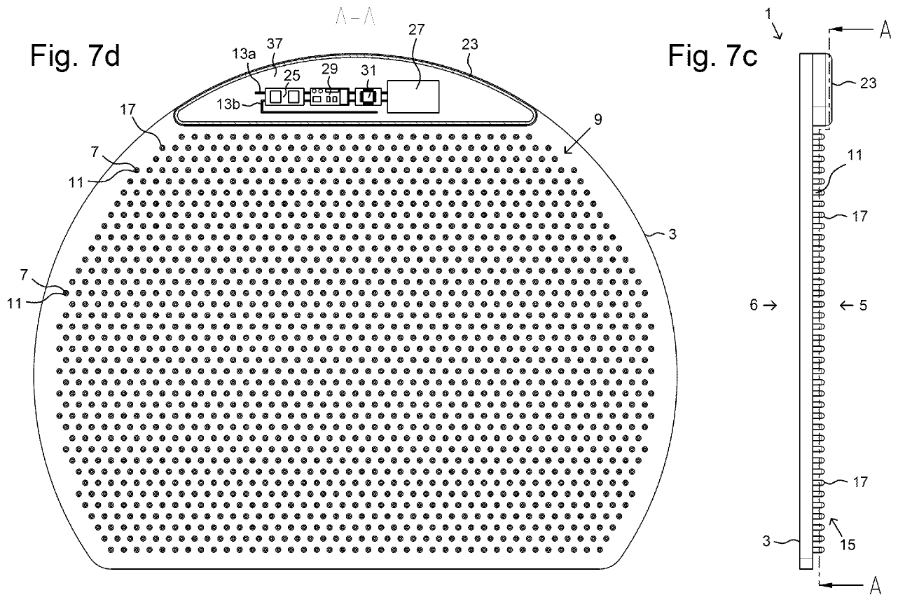

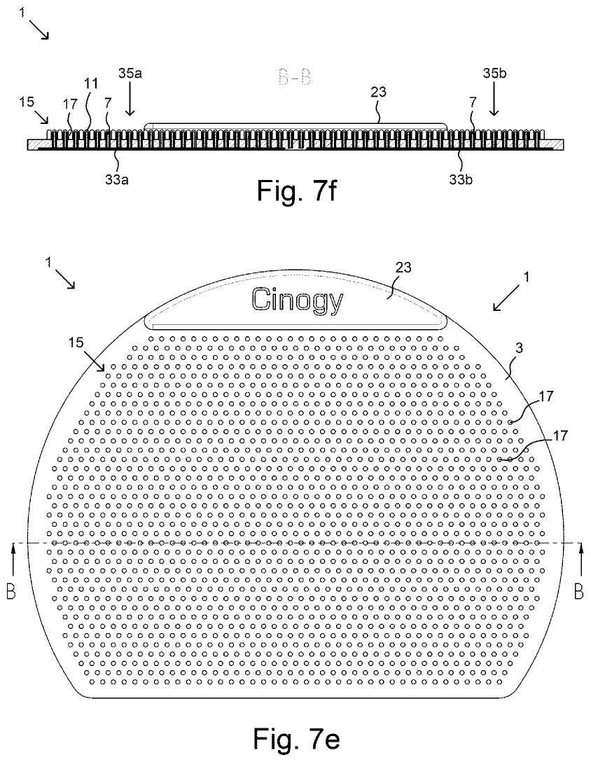

FIG. 7a shows a view of a treatment side of a fifth embodiment of the plasma treatment device according to the invention, in which the plasma treatment device is in the form of a floor mat;

FIG. 7b shows a side view of the fifth embodiment of the plasma treatment device;

FIG. 7c shows a further side view of the fifth embodiment of the plasma treatment device;

FIG. 7d shows a horizontal section along the line A-A in FIG. 7c;

FIG. 7e shows a further view of a treatment side of the fifth embodiment of the plasma treatment device;

FIG. 7f shows a vertical section along the line B-B in FIG. 7e.

In the figures, the same reference signs are used for elements that correspond to one another.

The exemplary embodiment shown in FIG. 1a shows a side view of a first embodiment of the plasma treatment device 1 according to the invention, which is designed to treat a surface with a dielectrically impeded. Further details of the structure of this first embodiment may be derived from FIGS. 1b, 1c, 1d, 1e, 1f and 1g.

In this first embodiment, the plasma treatment device is in the form of a curry comb and may be used, in this form, in particular to treat the hide of an animal.

It may be seen in FIG. 1a that the plasma treatment device 1 has a base body 3 that has a flat treatment side 5 facing the surface to be treated. In this exemplary embodiment, the base body 3 has a circular cross section and is made of an electrically insulating plastic.

It may furthermore be seen in particular in FIGS. 1a and 1b that a nub arrangement 15 is arranged on the treatment side 5 of the base body 3 and has a multiplicity of nubs 17. In this exemplary embodiment, the nub arrangement 15 is produced in one piece and formed completely from the dielectric 11. All of the nubs 17 of the nub arrangement 15 in this case have a circular cross section and are designed to be substantially circular-cylindrical with a rounded tip. The nubs 17 are of elongate design, wherein the ratio of the respective length L of the nubs to the longest dimension D of their respective cross section is greater than 3. In this first embodiment, the nub arrangement 15 and in particular the nubs 17, just like the dielectric 11 from which the nub arrangement 15 is formed, are designed to be flexible. This is achieved in that the dielectric 11—and thus the nub arrangement 15—are made from a flexible silicone.

It may furthermore be seen in particular in FIGS. 1a and 1c that the plasma treatment device 1 in this first embodiment has a handle 19, designed as a hand-operated handle, for holding the plasma treatment device 1 on a side facing away from the surface to be treated, specifically on a side opposite the treatment side 5. In this first embodiment, the plasma treatment device 1 is therefore particularly well-suited to manual treatment of an animal body and in particular to caring for and/or cleaning the hide of an animal.

It may furthermore be seen in particular from FIGS. 1a and 1c that the plasma treatment device 1 in this first embodiment has an electrically insulating housing 23 that is arranged on a side facing away from the surface to be treated, specifically on a side opposite the treatment side 5. The housing 23 is in this case designed in two parts and has a lower housing part 23a as well as an upper housing part 23b in the form of a cover and that is placed on the lower housing part 23a. In this exemplary embodiment, the lower housing part 23a is formed in this case by the base body 3 of the plasma treatment device 1.

FIG. 1e shows a sectional illustration of the first embodiment in the form of a horizontal section along the line B-B plotted in FIG. 1d, and thereby provides a view into the interior 37 of the housing 23. It may be seen in this case that the plasma treatment device in this exemplary embodiment has a high-voltage stage 25 arranged in the housing 23 and that is connected at output to a high-voltage supply line 13 that is likewise arranged in the housing 23. A battery 27, designed as a rechargeable battery (accumulator), is furthermore arranged in the housing 23 and is configured to provide a supply voltage required for the operation of the plasma treatment device 1. The plasma treatment device 1 in this exemplary embodiment—as in all of the following illustrative exemplary embodiments—is designed as a wireless device and is therefore able to be used in a particularly mobile and flexible manner. The housing 23 furthermore contains an electronic controller 31 and an inverter stage 29, by way of which an AC voltage signal is generated from a DC voltage supplied by the battery 27, which AC voltage signal is converted into a high-voltage signal in the form of an AC high-voltage signal by way of the high-voltage stage 25.

For this purpose, the electronic controller 31 controls the inverter stage 29 by way of which an AC voltage having an increased peak voltage is generated from the DC voltage of the battery 27 in a manner known per se, this peak voltage being able to be for example between 50 V and 500 V. The inverter stage 29, via its output, feeds the high-voltage stage 25, in which for example high-voltage pulses of 13 kV to 15 kV are generated.

FIG. 1f shows a vertical section along the line A-A in FIG. 1e. An electrode arrangement 9 that has a plurality of electrodes 7 is able to be seen here. In this exemplary embodiment, the electrodes 7 are of finger-shaped design and are surrounded by a dielectric layer of the dielectric 11. The dielectric 11 completely covers the electrodes 7 in the direction of the surface to be treated. The finger-shaped electrodes 7 each extend into a nub 17, formed from the dielectric 11, of the nub arrangement 15. In this exemplary embodiment, the electrodes 7 of the electrode arrangement 9 are designed to be flexible and the electrodes 7 are each partially embedded in the base body 3, wherein the electrodes 7 extending into a respective nub 17 protrude from the base body 3 on the treatment side 5 and protrude into a respective nub 17.

It may furthermore be seen from FIG. 1f that the electrode arrangement 9 comprising a plurality of electrodes 7 has an electrically conductive distributor plate 33 in this exemplary embodiment. The distributor plate 33 is connected firstly to the high-voltage supply line 13 of the line arrangement and connected secondly to all of the electrodes 7 of the electrode arrangement 9 and is configured to distribute the high-voltage signal to the electrodes 7 connected to the distributor plate 33. The electrodes 7 are thereby connected to the line arrangement comprising the high-voltage supply line 13 and are able to be supplied, via the high-voltage supply line 13, with the high-voltage signal able to be applied to the high-voltage supply line 13.

Between the nubs 17 of the nub arrangement 15 are air spaces in which the plasma is able to form during the treatment.

FIG. 1g shows a sectional illustration of the first embodiment in the form of a horizontal section along the line C-C in FIG. 1f. It may once again be seen in this case that the plasma treatment device 1 has a plurality of electrodes 7 having a circular cross section, which are covered by a dielectric layer of the dielectric 11 and thus shielded from the surface to be treated. The electrodes 7 each extend into a nub 17 of the nub arrangement 15. The nubs 17 and the electrodes 7 extending into the nubs are distributed over the circular cross section of the base body 3 and are in the process arranged on concentric circles.

It may be seen in this case from FIG. 1g that the electrodes 7 of the electrode arrangement 9 extend into a plurality of nubs 17 of the nub arrangement 15, but not into all of the nubs 17 of the nub arrangement 15. In this first embodiment, the nub arrangement 15, as shown by the horizontal section in FIG. 1g, specifically has both nubs 17 into which an electrode 7 of the electrode arrangement 9 extends and nubs 17 into which no electrode 7 extends. Those nubs 17 into which a respective electrode extends are in this case arranged in a manner distributed over the cross section of the base body 3 on concentric circles different from those nubs 17 into which no electrode 7 extends. The concentric circles containing nubs 17 into which electrodes 7 extend are in this case arranged alternately with concentric circles containing nubs 17 into which no electrodes 7 extend.

In the exemplary embodiment shown in FIGS. 1a to 1g, as in all of the exemplary embodiments shown in the following figures, the electrode arrangement 9 of the plasma treatment device 1 is configured such that the surface to be treated is used as counter-electrode.

FIG. 2a shows a side view of a second embodiment of the plasma treatment device 1 according to the invention, in which the plasma treatment device 1 is likewise in the form of a curry comb. Further details of the structure of this second embodiment may be derived from the sectional illustrations shown in FIGS. 2b, 2c, 2d and 2e.

The second embodiment shown here corresponds largely to the first embodiment, explained above, of the plasma treatment device 1. In contrast thereto, the line arrangement of the second embodiment shown here, as may be seen from FIG. 2b, however comprises a plurality of high-voltage supply lines, specifically a first high-voltage supply line 13a and a second high-voltage supply line 13b, to which different high-voltage signals are able to be applied. In this second embodiment, as is clear from FIGS. 2c and 2e, the electrode arrangement 9 furthermore comprises a plurality of electrodes 7 that form a plurality of electrode groups that are electrically insulated from one another. In the exemplary embodiment shown here, these are the two electrode groups 35a, 35b that are electrically insulated from one another. The electrodes 7 of the two electrode groups 35a, 35b are in this case arranged over the circular cross section of the base body 3 such that the electrodes 7 of the first electrode group 35a are arranged on a first cross-sectional half and the electrodes 7 of the second electrode group 35b are arranged on a second cross-sectional half. The two cross-sectional halves in this case each have the shape of semicircular surfaces and do not overlap. The electrodes 7 of the two electrode groups 35a, 35b may however also be arranged in a differently distributed manner in other exemplary embodiments.

It may furthermore be seen from the horizontal section shown in FIG. 2e that the electrode arrangement 9, in the second embodiment of the plasma treatment device 1, has a plurality of distributor plates that are electrically insulated from one another, specifically a first distributor plate 33a and a second distributor plate 33b. The electrodes 7 of the first electrode group 35a are in this case connected to the first distributor plate 33a and the electrodes 7 of the second electrode group 35b are connected to the second distributor plate 33b, as may be seen in the vertical section illustrated in FIG. 2c. The first distributor plate 33a is furthermore connected to the first high-voltage supply line 13a and the second distributor plate 33b is connected to the second high-voltage supply line 13b. The different electrode groups 35a, 35b are thereby able to be supplied with different high-voltage signals, that is to say the first electrode group 35a is able to be supplied with a first high-voltage signal via the first high-voltage supply line 13a and the second electrode group 35b is able to be supplied with a second high-voltage signal via the second high-voltage supply line 13b, wherein the second high-voltage signal may be different from the first high-voltage signal. For this purpose, the high-voltage stage 25 shown in FIG. 2b is configured to generate two different high-voltage signals that are each suitable for generating the plasma. For this purpose, the high-voltage stage 25 is connected at output firstly to the first high-voltage supply line 13a and secondly to the second high-voltage supply line 13b. The first high-voltage signal with which the first electrode group 35a is supplied and the second high-voltage signal with which the second electrode group 35b is supplied are in this case, in this exemplary embodiment, in the form of AC high-voltage signals that are in phase opposition to one another and that have a substantially identical amplitude.

For the rest, with regard to the second embodiment shown in FIGS. 2a to 2e, reference may be made to the explanations with regard to the first embodiment of the plasma treatment device 1 according to the invention shown in FIGS. 1a to 1g.

FIG. 3a shows a perspective view of a third embodiment of the plasma treatment device 1 according to the invention. In this third embodiment, the base body 3 is designed in the form of a glove, specifically in the form of a glove with fingers. It is possible to see a nub arrangement 15, which is arranged on the treatment side 5 of the base body 3 designed as a glove, is formed from the dielectric 11 and has a multiplicity of nubs 17 formed from the dielectric 11. In this exemplary embodiment too, the nub arrangement 15 is formed completely from the dielectric and is produced in one piece from a flexible silicone. The nub arrangement 15 is in this case produced as a separate part and is affixed to the base body 3 on the treatment side 5 with the production of a substance-to-substance bond, specifically by welding.

It may furthermore be seen from FIG. 3a that the plasma treatment device, in the third embodiment shown here as well, has an electrically insulating housing 23 that is made of a stable plastic and is arranged on a side opposite the treatment side 5. The housing 23 is connected to the nub arrangement 15 via a connecting piece 39 made of an insulating material and in which a high-voltage supply line 13 runs.

FIG. 3b shows a sectional illustration of the third embodiment of a vertical section along the line A-A in FIG. 3c. It is clear from the sectional illustration that the plasma treatment device, in this third embodiment as well, has an electrode arrangement 9 having a plurality of finger-shaped electrodes 7, wherein the electrodes 7 each extend into a nub 17 of the nub arrangement 15. The electrodes 7 are completely covered by the dielectric 11, from which the nubs 17 are formed, in the direction of the surface to be treated. The electrodes 7 are furthermore able to be supplied, via a high-voltage supply line 13 that runs in the connecting piece 39 and that is part of a line arrangement, with a high-voltage signal able to be applied to the high-voltage supply line 13.

The housing 23 is, in this embodiment as well, designed in two parts and has a lower housing part 23a and an upper housing part 23b in the form of a cover and that is able to be placed on the lower housing part 23a.

In this third embodiment, as may be seen for example in FIG. 3b, the base body 3, designed as a glove with fingers, furthermore has an insertion opening 21 through which a human hand is able to be inserted into the glove. To perform the treatment, a hand is inserted into the glove-shaped base body 3, and the plasma treatment device 1 is guided over the surface to be treated by way of the hand inserted into the base body 3.

Further details of the third embodiment of the plasma treatment device 1 according to the invention may be seen from the sectional illustration, shown in FIG. 3c, in the form of a horizontal section along the line B-B in FIG. 3b. It may be seen in particular that the plasma treatment device 1 has at least one battery, specifically three batteries 27 that are arranged in the housing 23, designed as accumulators and configured to provide a supply voltage required for the operation of the plasma treatment device 1. In the same way as the exemplary embodiments of the first and second embodiments explained above, the plasma treatment device furthermore has an electronic controller 31 arranged in the housing 23, an inverter stage 29 and a high-voltage stage 25. To this end, owing to the similarities in this regard, reference may be made to the explanations with regard to the exemplary embodiments of the first and second embodiment, which are shown in FIGS. 1a to 1 g and 2a to 2e.

FIG. 4a shows a perspective view of a fourth embodiment of the plasma treatment device according to the invention. Further details of this fourth embodiment may be derived from the sectional illustrations shown in FIGS. 4b, 4c, 4d and 4e.

In this fourth embodiment as well, the base body 3 is designed in the form of a glove with fingers. For the rest as well, the fourth embodiment shown in FIGS. 4a to 4e largely corresponds to the previously explained third embodiment of the plasma treatment device 1 according to the invention. In this respect, reference may therefore be made to the explanations with regard to the third embodiment explained above.

In contrast to the third embodiment explained above, the plasma treatment device according to the invention in the fourth embodiment shown here however has a plurality of electrodes 7 that form a plurality of electrode groups that are electrically insulated from one another, specifically form two electrode groups 35a, 35b that are insulated from one another. This may be seen in FIG. 4a in that the fourth embodiment, in contrast to the third embodiment, has not just one connecting piece 39 but two connecting pieces 39, in each of which one of two high-voltage supply lines 13a, 13b is arranged, different high-voltage signals being able to be applied to said high-voltage supply lines. The first electrode group 35a is in this case connected to the first high-voltage supply line 13a and the second electrode group 35b is connected to the second high-voltage supply line 13b. In this regard, reference may be made to the explanations with regard to the second embodiment shown in FIGS. 2a to 2e.

FIGS. 5a and 5b respectively show components of the third embodiment of the plasma treatment device according to the invention in a side view and in a view of a treatment side. For the sake of illustration, the plasma treatment device 1 is in this case shown without the dielectric 11 covering the electrodes 7 in the direction of the surface to be treated and without the nub arrangement 15.

It is thereby possible to see the electrode arrangement 9, which has a plurality of finger-shaped electrodes 7 that extend into the nubs 17 (not shown).

It may furthermore be seen in particular from FIG. 5b that the electrode arrangement, in this third embodiment of the plasma treatment device 1 according to the invention as well, has an electrically conductive distributor plate 33 that is connected to the high-voltage supply line 13 and all of the electrodes 7. In the same way as the first and second embodiment explained above, the distributor plate 33 is configured to distribute the high-voltage signal generated by the high-voltage stage 25 to the electrodes 7 connected to the distributor plate 33. In order to allow the base body 3 to be flexibly adapted to the surface to be treated, the distributor plate 33 in this third embodiment, like the base body 3, is designed to be flexible.

FIGS. 6a and 6b, in an illustration form corresponding to FIGS. 5a and 5b, shows components of the fourth embodiment of the plasma treatment device 1 according to the invention. It may be seen in this case, as already previously explained above, that the line arrangement of the plasma treatment device 1 in this fourth embodiment not only comprises two high-voltage supply lines 13a, 13b to which different high-voltage signals are able to be applied, but that the electrode arrangement 9 furthermore comprises a plurality of electrodes 7 that form two electrode groups 35a, 35b that are electrically insulated from one another. In the same way as the previously explained second embodiment, the two electrode groups 35a, 35b are in this case connected to different high-voltage supply lines 13a, 13b and are able to be supplied with different high-voltage signals.

It may furthermore be seen from the illustration in FIGS. 6a and 6b that the electrode arrangement 9, in this fourth embodiment, has two distributor plates 33a, 33b that are electrically insulated from one another, wherein the electrodes 7 of the two different electrode groups 35a, 35b are connected to different distributor plates 33a, 33b and the different distributor plates 33a, 33b are connected to different high-voltage supply lines 13a, 13b. The structure of the plasma treatment device 1 in this fourth embodiment corresponds to the structure of the second embodiment shown in FIGS. 2a to 2e with regard to the plurality of high-voltage supply lines, the plurality of electrode groups electrically insulated from one another and the plurality of distributor plates electrically insulated from one another, meaning that, in this respect, reference may be made to the explanations in this regard.

FIG. 7a shows a view of a treatment side of a fifth embodiment of the plasma treatment device 1 according to the invention, in which the plasma treatment device 1 is designed as a floor mat on which at least one foot is placed. FIG. 7b shows a side view of this fifth embodiment of the plasma treatment device 1.

It may be seen in FIGS. 7a and 7b that the plasma treatment device 1, in this fifth embodiment as well, has a base body 3 that has a flat treatment side 5 facing the surface to be treated and on which a nub arrangement 15 having a plurality of nubs 17 is arranged. The nubs 17 of the nub arrangement 15 are arranged in a manner distributed over the treatment side 5 of the base body 3. The base body 3 is made of a flexible plastic. The nub arrangement 15, in this exemplary embodiment as well, is designed in one piece and formed completely from the dielectric 11. The dielectric 11, and therefore also the nub arrangement 15, are in this case made from a flexible silicone.

The plasma treatment device 1 in this fifth embodiment is furthermore designed to be placed on a surface with a bottom side 6, which is a side opposite the treatment side 5. The bottom side 6 of the plasma treatment device 1, which is formed by a bottom side of the base body 3 in this embodiment, is designed to be non-slip in this exemplary embodiment. It is thereby possible to place the plasma treatment device 1 in this fifth embodiment on a surface, which may be for example a floor surface, and to use the plasma treatment device 1 in the manner of a foot massage mat that is known per se. For this purpose, one or two feet of a human to be treated or of an animal to be treated may be placed on the treatment side 5 on the plasma treatment device 1. A plasma treatment of the at least one foot to be treated may thereby be combined with a mechanical treatment of the foot in order to achieve a massage effect that emanates from the nubs 17 of the nub arrangement 15.

It may furthermore be seen in FIGS. 7a and 7b that the plasma treatment device 1 has an electrically insulating housing 23 that is made of a stable plastic. In this fifth embodiment, the housing 23 is arranged next to the nub arrangement 15 on the treatment side 5 of the base body 3.

FIG. 7c shows a further side view of the fifth embodiment of the plasma treatment device 1, and FIG. 7d shows a sectional illustration of the fifth embodiment in the form of a horizontal section along the line A-A in FIG. 7c. It may be seen in the sectional illustration of FIG. 7d that the plasma treatment device 1, in this embodiment as well, has an electrode arrangement 9 having a multiplicity of electrodes 7 that are surrounded by the dielectric 11 forming the nubs 17, such that the dielectric 11 completely covers the electrodes 7 in the direction of the surface to be treated. Both the nubs 17 and the electrodes 7 have a circular cross section in this exemplary embodiment.

It may furthermore be seen in FIG. 7d that a battery 27 for providing a supply voltage required for the operation of the plasma treatment device, as well as an electronic controller 31, an inverter stage 29 and a high-voltage stage 25, are arranged in an interior 37 of the housing 23. The operation of these components in this case corresponds to the previously explained embodiments, such that reference may be made to the explanations in this regard. A respective part of two high-voltage supply lines 13a, 13b is furthermore arranged in the housing 23. The electrodes 7 are connected to the high-voltage stage 25 via the high-voltage supply lines 13a, 13b, such that the electrodes 7 are able to be supplied with high-voltage signals via the high-voltage supply lines 13a, 13b. In this regard too, reference may also be made for the rest to the explanations with regard to the other embodiments of the plasma treatment device 1 according to the invention, in particular to the explanations with regard to the second and fourth embodiment.

FIG. 7e shows a further view of a treatment side 5 of the fifth embodiment of the plasma treatment device 1, and FIG. 7f shows a sectional illustration of this fifth embodiment in the form of a vertical section along the line B-B in FIG. 7e.

It may be seen in this case in the sectional illustration of FIG. 7f that the electrodes 7 have a finger-shaped design and extend into the nubs 17 of the nub arrangement 15. The electrodes 7 are in this case surrounded by a dielectric layer of the dielectric 11 that shields the electrodes in the direction of the surface to be treated.

It may furthermore be seen from FIG. 7f that the electrodes 7 form two electrode groups 35a, 35b that are electrically insulated from one another and that the electrode arrangement 9 has two distributor plates 33a, 33b that are electrically insulated from one another. The first electrode group 35a is in this case connected to the first distributor plate 33a and the second electrode group 35b is connected to the second distributor plate 33b. The different distributor plates 33a, 33b are furthermore connected to different high-voltage supply lines 13a, 13b, such that the first distributor plate 33a is connected to the first high-voltage supply line 13a and the second distributor plate 33b is connected to the second high-voltage supply line 13b. The different electrode groups 35a, 35b are thereby connected to different high-voltage supply lines 13a, 13b and are thus able to be supplied with different high-voltage signals, for example with AC high-voltage signals in phase opposition to one another. In this regard, reference may likewise be made to the explanations with regard to the other embodiments, in particular to the second and fourth embodiment.

### LIST OF REFERENCE SIGNS

1 plasma treatment device
3 base body
5 treatment side
6 bottom side
7 electrode
9 electrode arrangement
11 dielectric
13, 13a, 13b high-voltage supply line
15 nub arrangement
17 nub
19 handle
21 insertion opening
23 housing
23a lower housing part
23b upper housing part
25 high-voltage stage
27 battery
29 inverter stage
31 electronic controller
33, 33a, 33b distributor plate
35a, 35b electrode group
37 interior of the housing
39 connecting piece
L length of the nubs
D longest dimension of the cross section of the nubs

1. A plasma treatment device designed to treat a surface with a dielectrically impeded plasma, comprising:
a base body that comprises
at least one flat treatment side facing the surface to be treated,
an electrode arrangement that comprises
at least one electrode, and
a dielectric that completely covers the at least one electrode in a direction of the surface to be treated, and
a line arrangement comprising at least one high-voltage supply line, wherein the at least one electrode is connected to the line arrangement for supply by the at least one high-voltage supply line, with a high-voltage signal able to be applied to the at least one high-voltage supply line, and
a nub arrangement arranged on the at least one flat treatment side of the base body, wherein the nub arrangement has a multiplicity of nubs,
wherein the at least one electrode of the electrode arrangement extends into at least one nub of the nub arrangement.

2. The plasma treatment device as claimed in claim 1, wherein at least one of the nub arrangement and the base body are formed partially or completely from the dielectric.

3. The plasma treatment device as claimed in claim 1, wherein the nub arrangement is designed in one piece and/or the nub arrangement is designed in one piece with the base body.

4. The plasma treatment device as claimed in claim 1 wherein the at least one electrode of the electrode arrangement consists of a castable plastic provided with conductive additives.

5. The plasma treatment device as claimed in claim 1 wherein the at least one electrode of the electrode arrangement is flexible.

6. The plasma treatment device as claimed in claim 1 wherein the electrode arrangement is partially embedded in the base body, wherein the at least one electrode of the electrode arrangement that extends into at least one nub of the nub arrangement protrudes from the base body on the treatment side.

7. The plasma treatment device as claimed in claim 1 wherein the at least one electrode of the electrode arrangement comprises a plurality of finger-shaped electrodes that are surrounded by a dielectric layer and each of the plurality of finger-shaped electrodes extend into a separate nub of the multiplicity of nubs.

8. The plasma treatment device as claimed in claim 1 further comprising at least one handle and/or at least one strap for holding the plasma treatment device on a side facing away from the surface to be treated.

9. The plasma treatment device as claimed in claim 1 wherein the plasma treatment device is configured in a form of a curry comb.

10. The plasma treatment device as claimed in claim 1 wherein plasma treatment device is designed as a body part support mat on which at least one body part is placed.

11. The plasma treatment device as claimed in claim 1 wherein the base body is configured as a glove and has an insertion opening through which a human hand is able to be inserted into the glove.

12. The plasma treatment device as claimed in claim 1 further comprising an electrically insulating housing in which at least part of the at least one high-voltage supply line of the line arrangement is arranged.

13. The plasma treatment device as claimed in claim 12, wherein the electrically insulating housing
is arranged next to the nub arrangement on the treatment side of the base body, or
is arranged on a side facing away from the surface to be treated.

14. The plasma treatment device as claimed in claim 1 further comprising a high-voltage stage for generating high-voltage signals suitable for generating a plasma and that is connected at an output to the at least one high-voltage supply line of the line arrangement.

15. The plasma treatment device as claimed in claim 1 further comprising at least one battery for providing a supply voltage required for the operation of the plasma treatment device.

16. The plasma treatment device as claimed in claim 1 wherein the at least one electrode of the electrode arrangement comprises a plurality of electrodes, wherein the electrode arrangement has at least one electrically conductive distributor plate connected to the high-voltage supply line and plurality of electrodes, and wherein the at least one electrically conductive distributor plate is configured to distribute the high-voltage signal to one or more of the plurality of electrodes connected to the distributor plate.

17. The plasma treatment device as claimed in claim 1 wherein the at least one high voltage supply line of the line arrangement comprises a plurality of high-voltage supply lines to which different high-voltage signals are able to be applied, and wherein the at least one electrode of the electrode arrangement comprises a plurality of electrodes that form a plurality of electrode groups that are electrically insulated from one another, wherein different electrode groups are connected to different high-voltage supply lines and are able to be supplied with different high-voltage signals.

18. The plasma treatment device as claimed in claim 16 wherein the at least one electrically conductive distributor plate includes a plurality of distributor plates, and wherein the electrode arrangement has the plurality of distributor plates electrically insulated from one another, and wherein the at least one electrode of the electrode arrangement comprises a plurality of electrodes that form a plurality of electrode groups that are electrically insulated from one another, wherein the electrodes of the different electrode groups are connected to different distributor plates and the different distributor plates are connected to different high-voltage supply lines.

19. The plasma treatment device of claim 10 wherein the body part is at least one foot or at least one hand, and the body support mat is a floor mat or hand support mat, respectively.

20. The plasma treatment device of claim 11 wherein the glove has fingers.
